(19) United States
(12) Patent Application Publication
(10) Pub. No.: US 2020/0222039 A1
(43) Pub. Date: Jul. 16, 2020

(54) MEDICAL DEVICE FOR USE IN THE CREATION OF A TEMPORARY PNEUMOPERITONEUM

(71) Applicant: Dome Medical Technologies, Inc., New York, NY (US)

(72) Inventor: Glenn S. Foley, Vancouver, WA (US)

(21) Appl. No.: 16/837,837

(22) Filed: Apr. 1, 2020

Related U.S. Application Data
(63) Continuation-in-part of application No. 16/031,854, filed on Jul. 10, 2018.
Application No. 16/031,854 is a continuation-in-part of application No. 15/310,347, filed on Nov. 10, 2016, now abandoned.

(30) Foreign Application Priority Data
May 16, 2014 (GB) 1408740.7

(51) Int. Cl. A61B 17/02

(52) U.S. Cl. CPC A61B 17/0281; A61B 2017/00566

(57) ABSTRACT

A medical device for use in the creation of a temporary pneumoperitoneum includes a substantially dome-shaped body having a vacuum port providing a fluid passageway between an underside of the dome-shaped body and an upside of the dome-shaped body. The medical device includes a first grasping wing and a second grasping wing circumferentially opposite one another. The combination of the first grasping wing and the second grasping wing is configured to enable a user to single-handedly place the medical device at a surface of a patient. The medical device includes a plug disposed within an aperture of the dome-shaped body. The plug includes a first frustoconical aperture facing an opposite direction than a second frustoconical aperture to form a substantially hour-glass shape. The plug is configured to provide a penetrable barrier from the outside surface of the hard-body dome to the inside surface of the hard-body dome.

### CROSS-REFERENCE TO RELATED APPLICATIONS

This present application is a Continuation-in-Part of U.S. Non-Provisional application Ser. No. 16/031,854 entitled “MEDICAL DEVICE FOR USE. IN THE CREATION OF A TEMPORARY PNEUMOPERITONEUM” filed on Jul. 10, 2018, which is a Continuation of U.S. Non-Provisional application Ser. No. 15/310,347 entitled “MEDICAL DEVICE FOR USE IN THE CREATION OF A TEMPORARY PNEUMOPERITONEUM” filed on Nov. 10, 2016, which is a National Stage Entry of PCT/EP2015/000997, filed on May 15, 2015. Each above-referenced application is hereby incorporated by reference for all purposes.

### BACKGROUND

This invention relates to a medical device for use in the creation of a temporary pneumoperitoneum.

A laparoscopic surgical procedure is often preferred to a laparotomy due to shorter recovery times and the reduced adverse impact that it has on the patient's wellbeing. As part of the laparoscopic surgical procedure, a temporary pneumoperitoneum is formed in the patient's abdomen to separate the skin, tissue and muscle from the organs in the abdominal cavity below. This is achieved by insufflating the patient's abdomen with an inert gas, usually carbon dioxide (CO2).

Before insufflating the patient's abdomen with the inert gas, the skin, subcutaneous tissue and muscle are separated from the organs in the abdominal cavity below by applying a vacuum to the patient's abdomen. Once separated, a gas delivery needle can be inserted into the resulting space between the organs and the skin, tissue and muscle and the inert gas can be pumped into that space to stabilize and maintain the temporary pneumoperitoneum.

Heretofore, a number of devices have been proposed for the application of a vacuum to the patient's abdomen. One such device is that described in US2008/0058851 in the name of Edelstein et al. Another such device is that described in U.S. Pat. No. 7,585,281 in the name of Nezhat et al. A third such device is that described in WO2011/128713 filed in the name of Medical Device International Limited and having the same inventor as the present application. Although these devices all possess advantageous aspects over alternative ways of separating the skin, subcutaneous tissue and muscle from the organs, there are also disadvantages to the known devices. Most importantly, the known devices restrict the movement of a medical apparatus inserted through the device into the patient's abdomen and allow little or no room for positional adjustment of the medical apparatus. Secondly, some of the known devices have a relatively complex construction which increases the cost of manufacture. As these devices are intended to be disposable, a low manufacturing cost is essential.

### BRIEF DESCRIPTION OF THE DRAWINGS

FIG. 1 is a perspective view of a medical device for use in the creation of a temporary pneumoperitoneum, according to an embodiment.

FIG. 2 is a side cross-sectional view of the medical device of FIG. 1, according to an embodiment.

FIG. 3 is a side cross-sectional view of a second embodiment of a medical device according to the invention, according to an embodiment.

FIG. 4 is a side cross-sectional view of a third embodiment of a medical device, according to an embodiment.

FIG. 5A is a side-view of a medical device for use in the creation of a temporary pneumoperitoneum, according to an embodiment.

FIG. 5B is a top-view of a medical device depicted in FIG. 5A, according to an embodiment.

FIG. 5C is a side cross-sectional view of a medical device depicted in FIGS. 5A-B, according to an embodiment.

FIG. 5D is a side cross-sectional view of a medical device depicted in FIGS. 5A-B, according to an embodiment.

FIG. 6A is a perspective view of a plug for use in a medical device depicted in FIGS. 5A-B, according to an embodiment.

FIG. 6B is a side cross-sectional view of the plug depicted in FIG. 6A and disposed in the medical device depicted in FIGS. 5A-B, according to an embodiment.

### DETAILED DESCRIPTION

Methods, devices and systems related to a medical device for use in the creation of a temporary pneumoperitoneum are disclosed herein will become better understood through a review of the following detailed description in conjunction with the figures. The detailed description and figures provide merely examples of the various embodiments described herein. Those skilled in the art will understand that the disclosed examples may be varied, modified, and altered and not depart from the scope of the embodiments described herein. Many variations are contemplated for different applications and design considerations; however, for the sake of brevity, the contemplated variations may not be individually described in the following detailed description.

Throughout the following detailed description, example embodiments of various methods, devices and systems for the creation of a temporary pneumoperitoneum are provided. Related elements in the example embodiments may be identical, similar, or dissimilar in different examples. For the sake of brevity, related elements may not be redundantly explained in multiple examples except to highlight dissimilar features. Instead, the use of a same, similar, and/or related element names and/or reference characters may cue the reader that an element with a given name and/or associated reference character may be similar to another related element with the same, similar, and/or related element name and/or reference character in an example embodiment explained elsewhere herein. Elements specific to a given example may be described regarding that particular example embodiment. A person having ordinary skill in the art will understand that a given element need not be the same and/or similar to the specific portrayal of a related element in any given figure or example embodiment in order to share features of the related element.

As used herein “same” means sharing all features and “similar” means sharing a substantial number of features or sharing materially important features even if a substantial number of features are not shared. As used herein “may” should be interpreted in the permissive sense and should not be interpreted in the indefinite sense. Additionally, use of “is” regarding embodiments, elements, and/or features should be interpreted to be definite only regarding a specific embodiment and should not be interpreted as definite regarding the invention as a whole. Furthermore, references to “the disclosure” and/or “this disclosure” refer to the entirety of the writings of this document and the entirety of the accompanying illustrations, which extends to all the writings of each subsection of this document, including the Title, Background, Brief description of the Drawings, Detailed Description, Claims, and Abstract.

Where multiples of a particular element are shown in a FIG., and where it is clear that the element is duplicated throughout the FIG., only one label may be provided for the element, despite multiple instances of the element being present in the FIG. Accordingly, other instances in the FIG. of the element having identical or similar structure and/or function may not be redundantly labeled. A person having ordinary skill in the art will recognize based on the disclosure herein redundant and/or duplicated elements of the same FIG. Despite this, redundant labeling may be included where helpful in clarifying the structure of the depicted example embodiments. As used herein regarding a list, “and” forms a group inclusive of all the listed elements. For example, an embodiment described as including A, B, C, and D is an embodiment that includes A, includes B, includes C, and also includes D. As used herein regarding a list, “or” forms a list of elements, any of which may be included. For example, an embodiment described as including A, B, C, or D is an embodiment that includes any of the elements A, B, C, and D. Unless otherwise stated, an embodiment including a list of alternatively-inclusive elements does not preclude other embodiments that include various combinations of some or all of the alternatively-inclusive elements. An embodiment described using a list of alternatively-inclusive elements includes at least one element of the listed elements. However, an embodiment described using a list of alternatively-inclusive elements does not preclude another embodiment that includes all of the listed elements. And, an embodiment described using a list of alternatively-inclusive elements does not preclude another embodiment that includes a combination of some of the listed elements. As used herein regarding a list, “and/or” forms a list of elements inclusive alone or in any combination. For example, an embodiment described as including A, B, C, and/or D is an embodiment that may include: A alone; A and B; A, B and C; A, B, C, and D; and so forth. The bounds of an “and/or” list are defined by the complete set of combinations and permutations for the list.

It is an aim of the present invention to provide a medical device for use in the creation of a temporary pneumoperitoneum that overcomes at least some of the problems with the known devices. It is a further aim of the present invention to provide a medical device for use in the creation of a temporary pneumoperitoneum that offers a useful alternative to the consumer.

According to the invention there is provided a medical device for use in the creation of a temporary pneumoperitoneum, the medical device comprising a substantially dome-shaped body having a vacuum port providing a fluid passageway between the underside of the dome-shaped body and the upside of the dome-shaped body, the dome-shaped body further comprising a frustoconical port for reception and through-passage of one or more pieces of medical apparatus therethrough.

The underside of the dome-shaped body is intended to mean the side closest to the patient's body, whilst the upside is intended to mean the side furthest from the patient's body.

As bodies vary from patient to patient, it is difficult to predict precisely how a medical device will settle on the patient's body, for example their abdomen, once the vacuum is applied. With the known devices, a degree of trial and error is sometimes required to ensure that the medical apparatus for insertion through the medical device will be correctly aligned with the part of the abdomen being targeted. In other cases, the medical apparatus will be positioned sub-optimally. By having a device according to the present invention with a frustoconical port, it will be possible for the surgical team to adjust the position and the orientation of the medical apparatus inserted through the frustoconical port of the medical device with relative ease. The direction of the medical apparatus can be adjusted from side to side through an arc of close to 180° and the distal tip of the medical apparatus can be rotated around in a circle through 360°, allowing for very precise placement of the medical apparatus. Furthermore, such adjustability will allow for more forgiving placement of the medical device on the patients abdomen. These will speed up the surgical procedure and greatly facilitate the performance of the surgical procedure.

The dome-shaped body will generally be constructed from a material that is suitably robust and rigid that can withstand significant vacuum pressures without collapsing inwardly onto the patient's abdomen. It is envisaged that the dome-shaped body can be constructed from a plastics material for this purpose, for example, a medical grade polycarbonate or equivalent material. The body is described throughout as being dome-shaped and indeed this is preferable for the spread of the vacuum induced forces on the body. However, other shapes of body are envisaged and may be put to good use instead of a strictly dome-shaped body.

In one embodiment of the invention there is provided a medical device in which the frustoconical port is closed. This is seen as a particularly useful aspect of the present invention. By having a closed port, the vacuum can be created faster and with greater ease. Secondly, the device will tend to settle more predictably on the patient's abdomen due to the fact that air is not being drawn into the device from another location on the dome-shaped body. Furthermore, there will be less chance of ingress of foreign bodies into the abdominal cavity.

In one embodiment of the invention there is provided a medical device in which the frustoconical port is closed with a plug. A plug may be inserted with ease into the frustoconical port to close the port during manufacture. The plug may be formed from a different material to the remainder of the dome-shaped body which can be advantageous for a number of reasons. For example, the plug can be constructed from a material that will be easier to puncture than the remainder of the dome-shaped body, thereby facilitating insertion of the medical apparatus through the port. Furthermore, the plug may be constructed from a material impregnated with an antibacterial agent to obviate the possibility of the medical apparatus being inserted into the patient causing an infection. Similarly, the plug may be impregnated with a local anaesthetic to numb the area of the patient's abdomen that comes into contact with the plug.

In one embodiment of the invention there is provided a medical device in which the plug comprises a penetrable barrier. By “penetrable”, what is meant is a barrier that it will be possible to penetrate with relative ease using a medical instrument such as a needle or the like. In a preferred embodiment, the penetrable material comprises a softer material than the dome-shaped body, for example, a semi-latex material. A semi-latex material is understood to be one which contains chains of latex polymer but do not cause hypoallergenic issues. The penetrable material may alternatively comprise a non-latex polymer material. In certain instances, the materials used for the dome-shaped body and the plug will be biodegradable and suitable for disposal after the device has been used. The manufacturing method will be highly cost-effective due in large part to the simplicity of the manufacturing process (a two-stage mould process) and this will contribute to a very cost-effective medical device.

In one embodiment of the invention there is provided a medical device in which the frustoconical port is a blind bore formed in the dome-shaped body. This is seen as a useful alternative as apart from the vacuum port, the dome-shaped body will not have any apertures through which air can pass. This will facilitate the creation of the vacuum and when necessary, the medical apparatus can be used to puncture through the dome-shaped body at the base of the blind bore.

In one embodiment of the invention there is provided a medical device in which there is provided a target located on the upside of the dome-shaped body coincident with and concentric with the frustoconical port. By providing a target, the user will be able to locate the frustoconical port with relative ease, thereby facilitating the operation of the device.

In one embodiment of the invention there is provided a medical device in which the thickness of the dome-shaped body at the base of the blind bore is less than or equal to 5 mm thick. By having the dome-shaped body no more than 5 mm thick at this point, the dome-shaped body can be punctured with relative ease. Ideally, the thickness of the dome-shaped body at the base of the blind bore is less than or equal to 1.5 mm thick.

In one embodiment of the invention there is provided a medical device in which the frustoconical port opens outwardly in the direction from the upside of the dome-shaped body to the underside of the dome-shaped body. This is seen as a preferred orientation of the frustoconical port as the opening at the upside of the dome-shaped body can still be relatively small which is good for particle ingress prevention and will allow for a more steady engagement of the medical apparatus passed through the frustoconical port in due course.

In one embodiment of the invention there is provided a medical device in which there is provided a second frustoconical port on the dome-shaped body located above and coincident with the first frustoconical port, the second frustoconical port opens outwardly in the direction from the underside of the dome-shaped body to the upside of the dome-shaped body, the pair of frustoconical ports thereby combining to form a substantially hourglass-shaped port for reception and through-passage of one or more pieces of medical apparatus therethrough.

In one embodiment of the invention there is provided a medical device in which the dome-shaped body further comprises another frustoconical port for reception and through-passage of one or more pieces of medical apparatus therethrough. Another frustoconical port will allow for other pieces of equipment to be inserted into the abdominal cavity from other directions, thereby facilitating certain surgical procedures.

In one embodiment of the invention there is provided a medical device in which the frustoconical port is located substantially centrally at the axis of the dome-shaped body. If another frustoconical port is provided it may be located on the side of the dome-shaped body spaced apart from the first frustoconical port.

In one embodiment of the invention there is provided a medical device in which there is provided an annular flange at the base of the dome-shaped body. The annular flange may be outwardly depending from the base of the dome-shaped body. The flange will allow for a secure seal to be formed between the base of the dome-shaped body and the patient's abdomen facilitating vacuum creation and retention and also reducing bruising to the patient's abdomen. Alternatively, the thickness of the dome-shaped body at the base could be dimensioned so that it performs the same function as the annular flange. Further still, the annular flange could be inwardly depending rather than outwardly depending.

In one embodiment of the invention there is provided a medical device in which the dome-shaped body is saucer dome-shaped. A saucer dome-shape may be more suited ergonomically to certain body types and may be easier for the patient's body to mould into.

Whilst reference has been made to a medical device for use in the creation of a temporary pneumoperitoneum in abdominal procedures, it will be understood that the medical device described herein may also be used in other areas where key-hole surgery is performed. The dome-shaped housing may be resized accordingly so that it will form a suitable seal with the patient's body around the area to be operated upon.

The invention will now be more clearly understood from the following description of some embodiments thereof given by way of example only and with reference to the accompanying drawings.

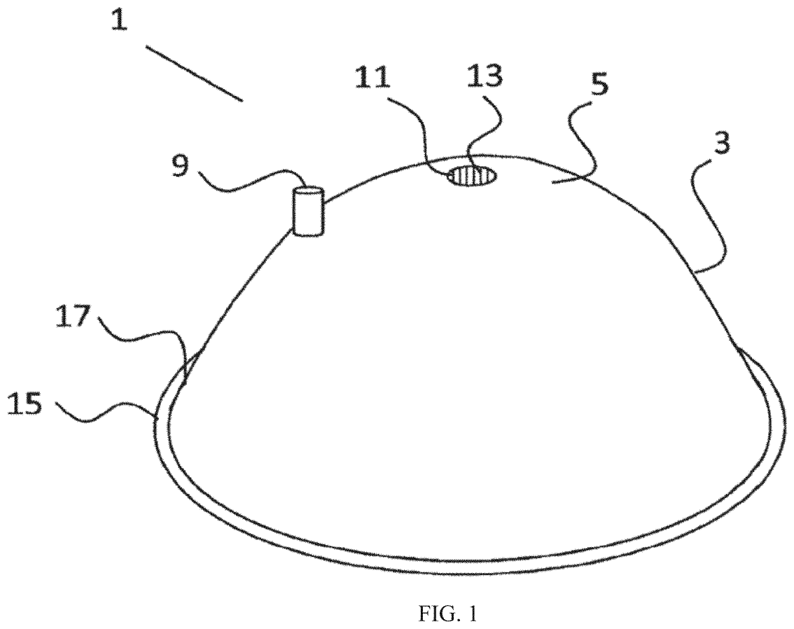

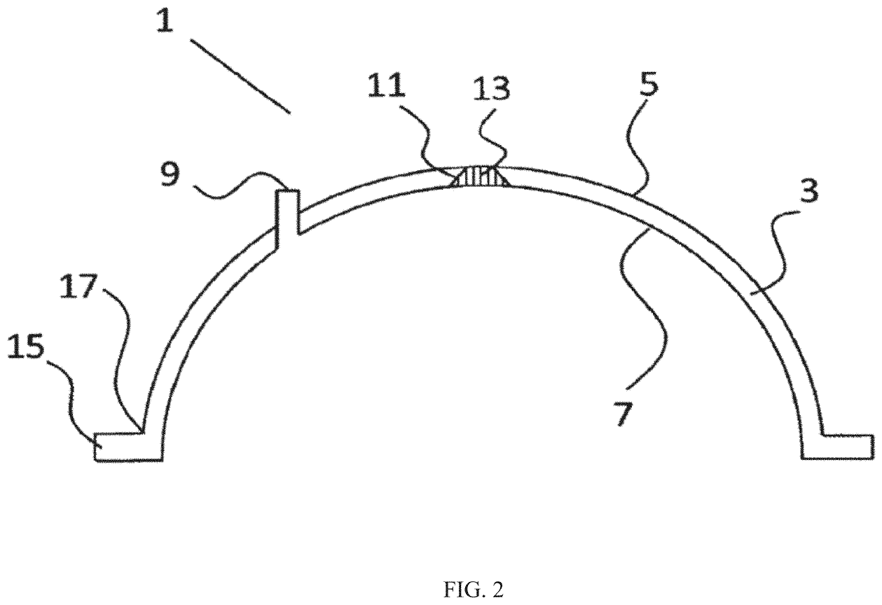

Referring to FIGS. 1 and 2, there is shown a medical device for use in the creation of a temporary pneumoperitoneum, indicated generally by the reference numeral 1, the medical device comprising a substantially dome-shaped body 3 having an upside 5, an underside 7, and a vacuum port 9 providing a fluid passageway between the upside 5 and the underside 7. The dome-shaped body 3 further comprises a port 11 for reception and through-passage of one or more pieces of medical apparatus (not shown) therethrough. The port 11 is frustoconically shaped with the port widening in the direction from the upside 5 to the underside 7. By having a frustoconical port 11, a medical apparatus (not shown) inserted therethrough may be directionally adjusted in order to allow the medical apparatus to be positioned more accurately without requiring adjustment of the entire medical device 1.

A plug 13 is mounted in the frustoconical port 11, thereby closing the port. The plug 13 is constructed from a penetrable barrier material that will allow a medical apparatus to be pushed therethrough. The dome-shaped body 3 has an annular flange 15 at the base 17 thereof. The annular flange 15 is outwardly depending from the base 17 of the dome-shaped body 3. Potentially, a suitable hypoallergenic covering may be provided on the underside of the dome-shaped body and more importantly on the underside of the annular flange.

In use, the medical device for use in the creation of a temporary pneumoperitoneum 1 is placed on the abdomen of a patient (not shown). The annular flange 15 forms a seal with the patient's abdomen. A vacuum/suction device is connected up to the vacuum port 9 and air is removed from between the underside 7 of the dome-shaped body 3 and the patient's abdomen. As the air is removed, the patient's abdomen will be drawn upwards towards the underside of the dome-shaped body. This has the effect of separating the skin, subcutaneous tissue and muscle from the organs in the abdominal cavity below. A needle (not shown) or similar device is then inserted through the plug 13 in the frustoconical port 11 and into the patient's abdomen. A camera may be mounted on the needle to allow the surgeon to monitor the location of the distal tip of the needle. Once the distal tip of the needle is in the desired location within the patient's abdomen, an inert gas, typically CO2, is delivered into the cavity in the patient's abdomen, thereby forming the temporary pneumoperitoneum. The desired surgical procedure is then performed.

The dome-shaped body 3 may be left in situ throughout the surgical procedure or alternatively it may be removed if desired. If the dome-shaped body is left in situ, the frustoconical port should be large enough to allow more than one medical apparatus therethrough to allow the operation to be performed. Once the operation is finished, the dome-shaped body 3 may be discarded.

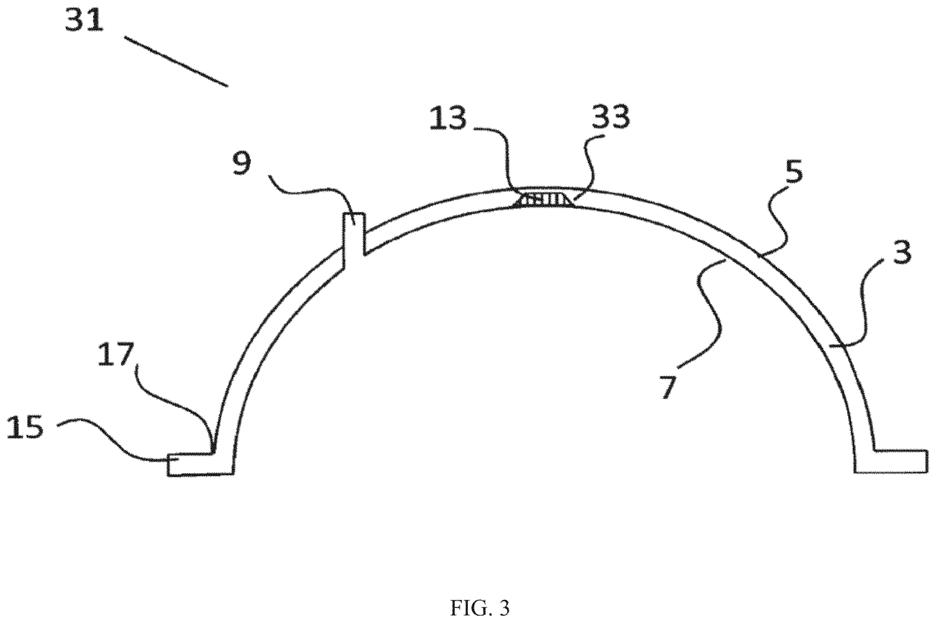

Referring to FIG. 3, there is shown an alternative embodiment of a medical device (second embodiment), indicated generally by the reference numeral 31, in which like parts have been given the same reference numeral as before. In the embodiment shown, the frustoconical bore 33 is a blind bore. In other words, the frustoconical bore 33 does not extend through the dome-shaped body. In this implementation, a target (not shown) will preferably be provided, either printed or scored into the upside of the dome-shaped body coincident and concentric with the frustoconical port 33. Again, a plug 13 is provided in the frustoconical bore 33. The thickness of the dome-shaped body at the base of the blind bore 33 will be of an appropriate thickness that will allow piercing by one or more of a plurality of medical devices. Preferably, the thickness of the dome-shaped body at the base of the blind bore will be of the order of 1.5 mm (1.5×10-3 m). By having the bore closed in this fashion, the vacuum can be created in a more effective manner.

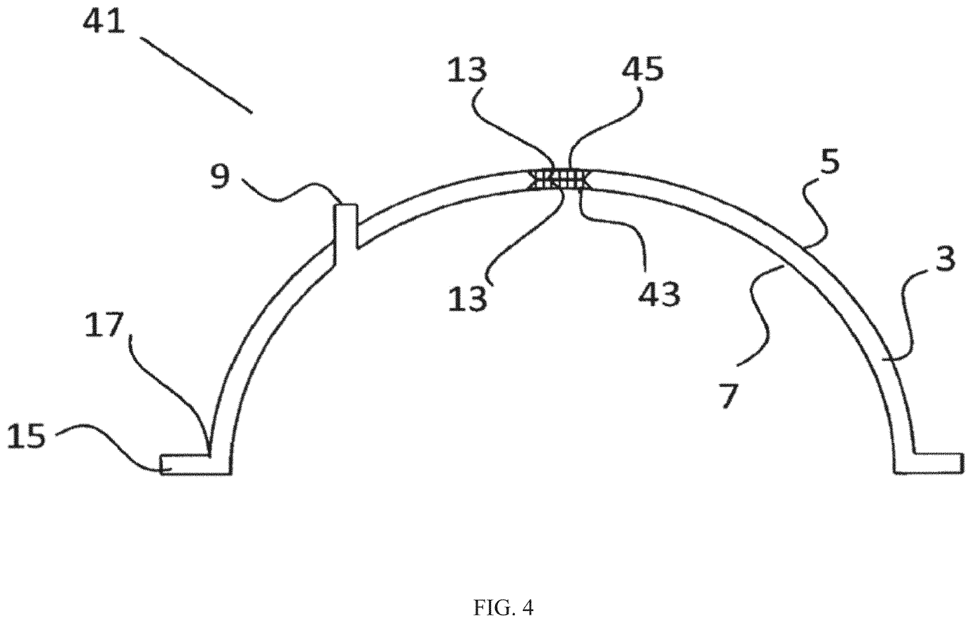

Referring to FIG. 4, there is shown a further alternative embodiment of a medical device (third embodiment), indicated generally by the reference numeral 41, in which like parts have been given the same reference numeral as before. In the embodiment shown, there is provided a second frustoconical port 45 located above and coincident with the first frustoconical port 43. The two frustoconical ports are arranged face to face so that together the two frustoconical ports 43, 45 form an hourglass-shaped port. If desired, the frustoconical ports 43, 45 may be blind bores separated by a dividing wall therebetween.

Various modifications could be made to the above described embodiments without departing from the scope of the present invention as defined in the claims. For example, another port could be provided elsewhere on the device 1, 31, 41 to allow a second medical apparatus to be inserted into the patient's cavity from an alternative angle than the first medical apparatus. In the embodiments shown, there is provided an annular flange 15 however if desired the thickness of the dome-shaped body at the base could be dimensioned so that it performs the same function as the annular flange. Further still, the annular flange could be inwardly depending rather than outwardly depending.

The dome-shaped body will generally be constructed from a material that is suitably robust and rigid that can withstand significant vacuum pressures without collapsing inwardly onto the patient's abdomen. It is envisaged that the dome-shaped body can be constructed from a plastics material for this purpose, for example, a medical-grade polycarbonate or equivalent material. The body is described throughout as being dome-shaped and indeed this is preferable for the spread of the vacuum induced forces on the body. However, other shapes of body are envisaged and may be put to good use instead of a strictly dome-shaped body.

The penetrable plug 13 will preferably be provided by a softer material than the dome-shaped body, for example, the penetrable plug could be constructed from latex, or more preferably from semi-latex or a non-latex polymer material. In certain instances, the materials used for the dome-shaped body and the plug will be biodegradable and suitable for disposal after the device has been used. The manufacturing method will be highly cost-effective due in large part to the simplicity of the manufacturing process (a two-stage mould process) and this will contribute to a very cost-effective medical device.

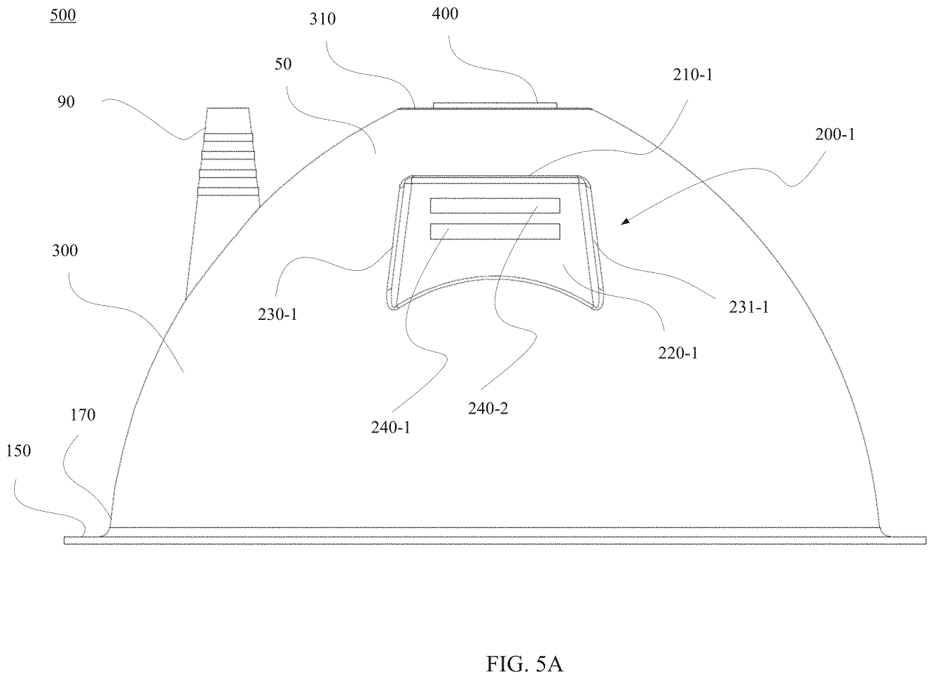

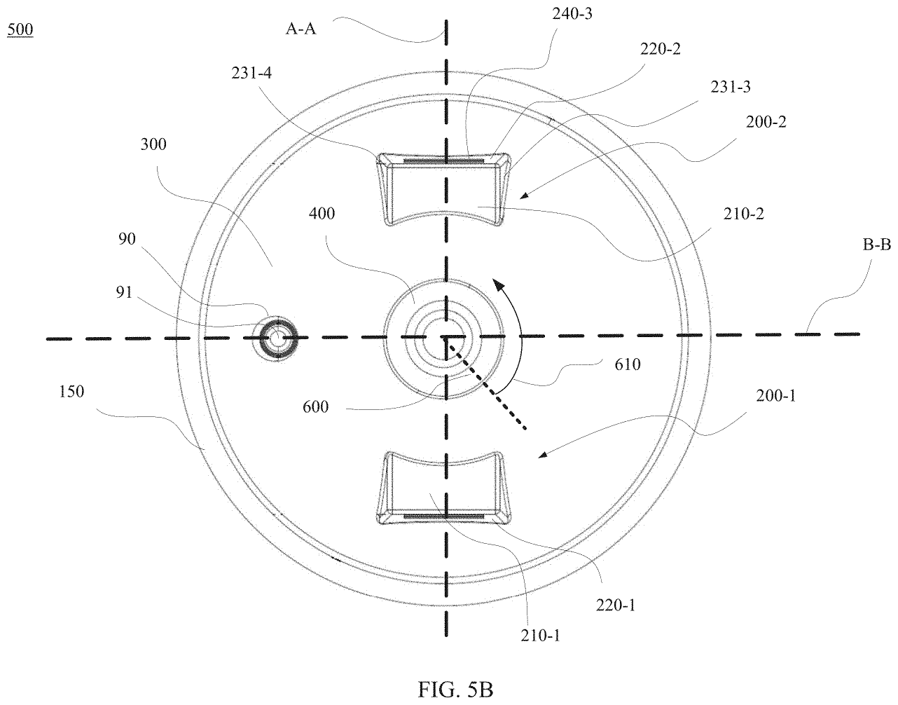

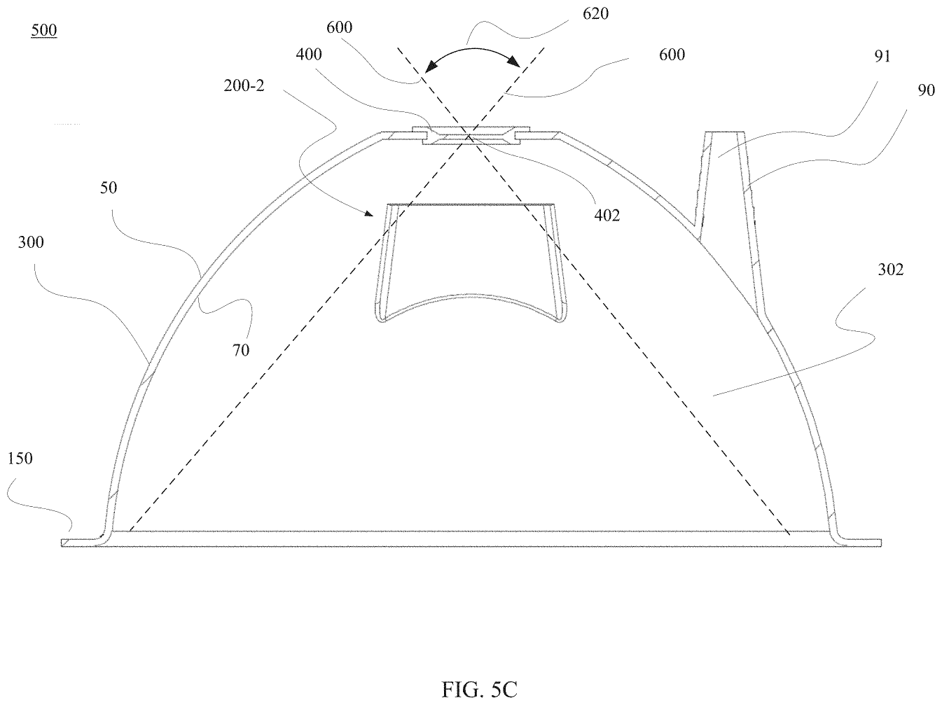

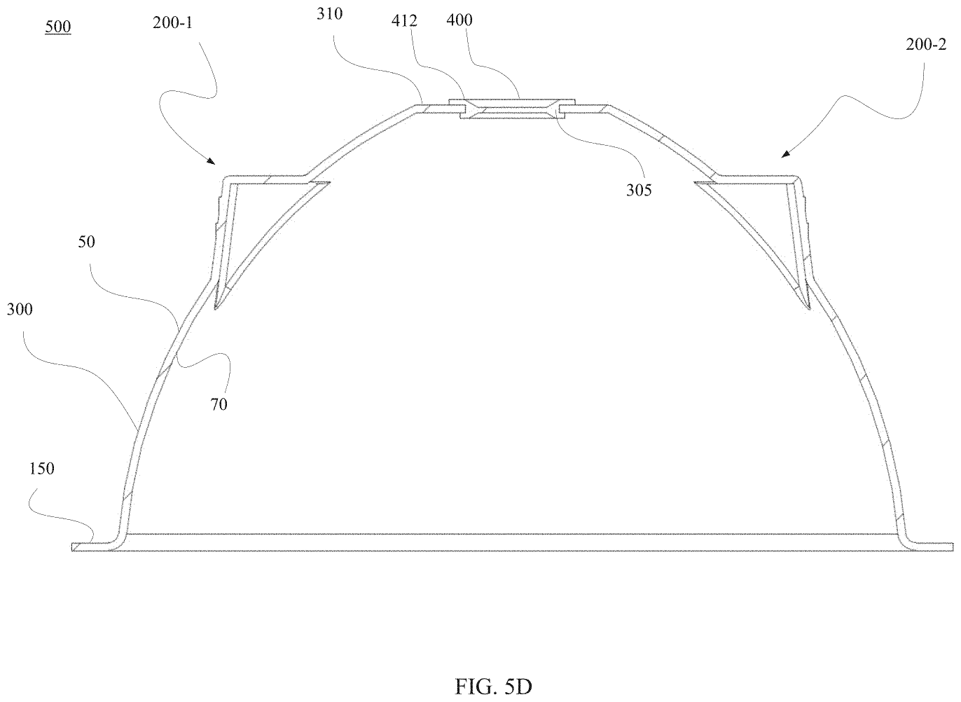

FIG. 5A depicts a side-view of a medical device 500 for use in the creation of a temporary pneumoperitoneum, according to an embodiment. Some of the features in FIG. 5A are the same as or similar to some of the features in FIGS. 1-4 as noted by same and/or similar reference characters, unless expressly described otherwise. Furthermore, the elements and/or features described regarding FIG. 5A may be the same as and/or similar to other similarly named elements and/or features described and/or illustrated throughout this disclosure. For example, the device 500 is similar to device 1 depicted in FIGS. 1 and 2, device 31 depicted in FIG. 3 and device 41 in FIG. 4.

The device 500 includes dome-shaped body 300 (also referred to herein as “dome 300” or “body 300”) that is similar to dome-shaped body 3. The dome 300 includes, among other things, an outer surface 50 (similar to outer surface 5), an annular flange 150 (similar to flange 15) disposed at a base 170 (similar to base 17) of dome 300, a vacuum feature 90 (similar to port 9) projecting from outer surface 50, a flat top surface (or apex) 310, a plug 400 (similar to plug 13) disposed in a through-hole at top surface 310, a first grasping wing 200-1 (also referred to herein as “wing 200-1”) projecting out of the outer surface 50.

The vacuum feature 90 may include a conical shape. The vacuum feature 90 may project vertically from outer surface 50. In one embodiment, a top surface of vacuum aperture is substantially co-planar with top surface 310.

The wing 200-1 includes an upper surface 210-1, an outer surface 220-1, a first side wall 230-1 and a second side wall 231-1. The upper surface 210-1 extends substantially horizontally from outer surface 50. The outer surface 220-1 extends substantially vertically downward from a distal end of upper surface 210-1.

In various embodiments, outer surface 220-1 includes a first grip feature 240-1 and second grip feature 240-2. In one embodiment, the grip features may protrude from outer surface 220-1. In another embodiment, the grip features are apertures that extend into the outer surface 220-1. The wing 210-1, and in particular, the grip features are configured to enable a user to grip the device 500 and properly place the device 500 at a location on a patient and remove the device from the location on the patient. In various embodiments, the grip features may have a curvature to conform to the shape of a finger of the user. Additionally, one or more grip features may be disposed on upper surface 210-1.

The plug 400 is configured to provide a penetrable barrier from the outer surface 50 of the dome 300 to the inside surface of the dome, and allow a medical apparatus to penetrate through the plug and access the surface of the patient disposed within the dome 300.

FIG. 5B is a top-view of a medical device 500 depicted in FIG. 5A, according to an embodiment. Some of the features in FIG. 5B are the same as or similar to some of the features in FIGS. 1-5A as noted by same and/or similar reference characters, unless expressly described otherwise. Furthermore, the elements and/or features described regarding FIG. 5B may be the same as and/or similar to other similarly named elements and/or features described and/or illustrated throughout this disclosure.

The dome 300 includes vacuum aperture 91 disposed within vacuum feature 90. The vacuum aperture 91 extends from an upper surface of vacuum feature 90 to an inner surface of dome 300. Accordingly, the vacuum aperture 91 is configured to provide a fluid passageway from within the dome 300 to an outside of the dome 300.

The dome 300 includes a wing 200-2 that is circumferentially opposite wing 200-1. The wing 200-2 may be similar to wing 200-1. In various embodiments, wing 200-2 includes upper surface 210-2 and outer surface 220-2. The upper surface 210-2 may extend horizontally from outer surface 50. The outer surface 220-2 extends substantially vertically downward from a distal end of upper surface 210-2. In various embodiments, the outer surface 220-2 includes grip features 240-3 similar to grip features 240-1 and 240-2 of wing 200-1. The grip features 240-3 are configured to enable a user to grip the device 500 and properly place the device 500 at a location on a patient and remove the device from the location on the patient.

In various embodiments, the wing 200-1 and the wing 200-2 are configured to enable a user to place dome 300, by a single hand of the user, at a surface of a patient (e.g., stomach). For example, a first digit of a hand (e.g., thumb) applies downward force on the wing 200-1 and a second digit (e.g., pointer finger) of the hand concurrently applies a downward force on the wing 200-2. The downward force may be applied to respective upper surfaces of the wings or on the respective outer surfaces of the wings.

In various embodiments, the wing 200-1 and the wing 200-2 are configured to enable a user to remove the dome 300, by a single hand of the user, from the surface of a patient (e.g., stomach). For example, a first digit of a hand (e.g., thumb) applies an upward force on the wing 200-1 and a second digit (e.g, pointer finger) of the hand concurrently applies an upward force on the wing 200-2. The upward force may be applied to the respective outer surfaces of the wings.

In one embodiment, a plane (e.g., plane A-A) bisects wing 200-1, plug 400 and wing 200-2. Additionally, the vacuum feature 90 may be laterally offset from the plane.

In various embodiments, a medical instrument 600 (e.g., a gas needle) penetrates plug 400 and is disposed partially within dome 300 and partially outside of dome 300. Accordingly, a user is able to move the medical instrument 600 within dome 300 by manually manipulating the medical instrument 600 that is disposed outside of dome 300. In one embodiment, the medical instrument 600 is able to be circumferentially rotated (e.g.,)360° about an insertion point of the plug 400 as indicate by arrow 610.

FIG. 5C is a side cross-sectional view of a medical device 500, according to an embodiment. In particular, the cross-sectional view of medical device 500 is along plane B-B (as shown in FIG. 5B). Some of the features in FIG. 5C are the same as or similar to some of the features in FIGS. 1-5B as noted by same and/or similar reference characters, unless expressly described otherwise. Furthermore, the elements and/or features described regarding FIG. 5C may be the same as and/or similar to other similarly named elements and/or features described and/or illustrated throughout this disclosure.

The dome 300 includes an inner surface 70 opposite outer surface 50. The medical instrument 600 penetrates plug 400 at insertion point 402. Accordingly, a portion of the medical instrument 600 is inserted into an inner portion 302 of dome 300. In various embodiments, the medical instrument 600 is able to pivot about insertion point 402. For example, the medical instrument 600 is depicted at a first position (at a first angulation) and at a second position (at a second angulation). In one embodiment, the range of angulation, as depicted by arrow 620, may be 150°. In another embodiment, the range of angulation may be more than 150° (e.g., 170°) or less than 150° (e.g., 130°).

In one embodiment, the range of angulation of the medical instrument about insertion point 402 prohibits the medical instrument 600 from contacting the inner surface 70 of dome 300.

FIG. 5D is a side cross-sectional view of a medical device 500, according to an embodiment. In particular, the cross-sectional view of medical device 500 is along plane A-A (as shown in FIG. 5B). Some of the features in FIG. 5D are the same as or similar to some of the features in FIGS. 1-5C as noted by same and/or similar reference characters, unless expressly described otherwise. Furthermore, the elements and/or features described regarding FIG. 5D may be the same as and/or similar to other similarly named elements and/or features described and/or illustrated throughout this disclosure.

As shown in FIG. 5D, in one embodiment, wing 200-1 is circumferentially opposite wing 200-2. In one embodiment, a top surface of 412 of plug 400 is vertically offset from top surface 310 of dome 300.

Additionally, top surface 310 includes a through-hole 305. In various embodiments, plug 400 is releasably seated within through-hole 305.

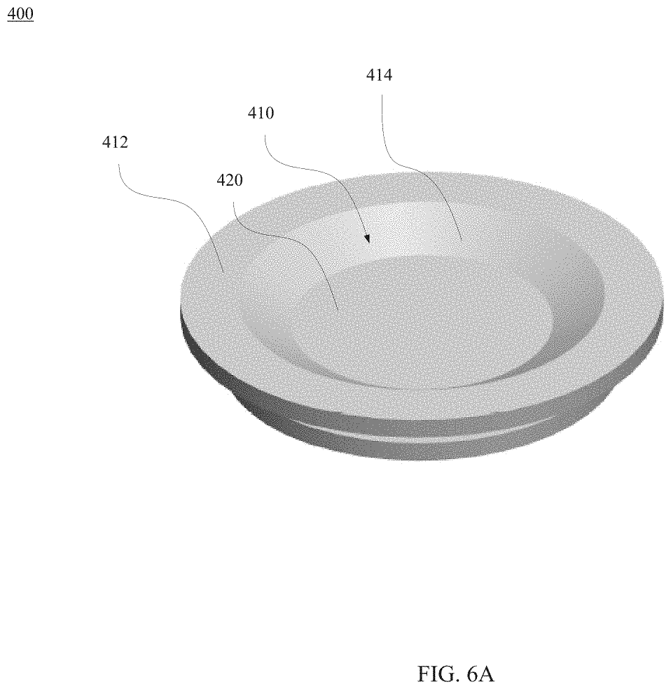

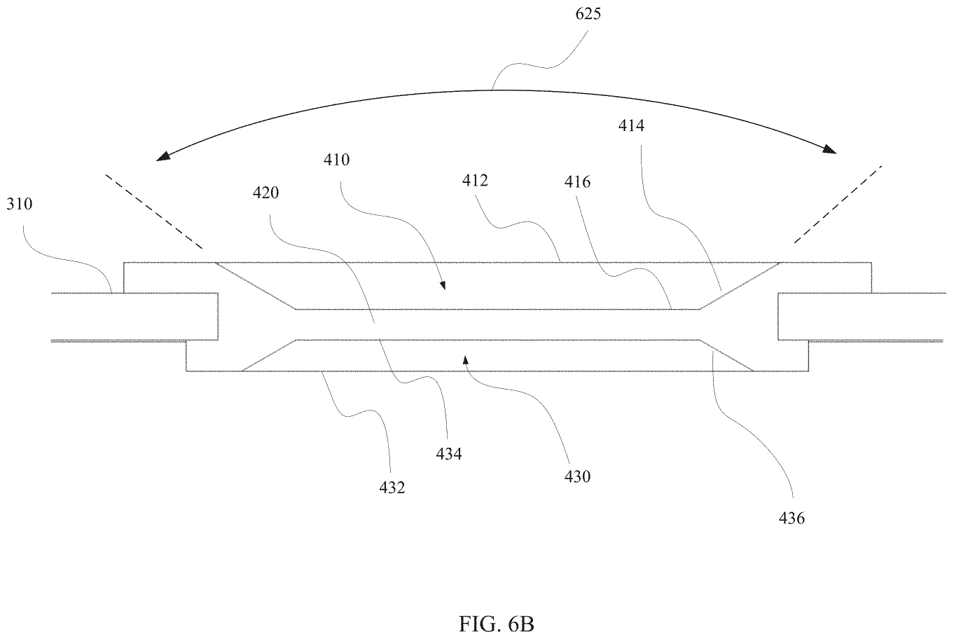

FIG. 6A is a perspective view of plug 400, according to an embodiment. Some of the features in FIG. 6A are the same as or similar to some of the features in FIGS. 1-5D as noted by same and/or similar reference characters, unless expressly described otherwise. Furthermore, the elements and/or features described regarding FIG. 6A may be the same as and/or similar to other similarly named elements and/or features described and/or illustrated throughout this disclosure.

The plug 400 includes top surface 412, a first frustoconical aperture 410 (also referred to herein as a frustoconical port), an intermediary wall 420, and a side wall 414.

The first frustoconical aperture 410 extends from the intermediary wall 420 to top surface 412. Accordingly, the frustoconical aperture 410 opens “upward” in the direction from the intermediary wall 420 to the top surface 412.

FIG. 6B is a side cross-sectional view of the plug 400 depicted in FIG. 6A and disposed within dome 300, according to an embodiment. Some of the features in FIG. 6B are the same as or similar to some of the features in FIGS. 1-6A as noted by same and/or similar reference characters, unless expressly described otherwise. Furthermore, the elements and/or features described regarding FIG. 6B may be the same as and/or similar to other similarly named elements and/or features described and/or illustrated throughout this disclosure.

The plug 400 includes top surface 412, a bottom surface 432, a first frustoconical aperture 410, an intermediary wall 420, and a side wall 414. The first frustoconical aperture 410 extends from the intermediary wall 420 to top surface 412.

The plug 400 includes a second frustoconical aperture 430. The second frustoconical aperture 430 extends from the intermediary wall 420 to bottom surface 432. Accordingly, the second frustoconical aperture 430 opens “downward” in the direction from the intermediary wall 420 to the bottom surface 432. Moreover, the second frustoconical aperture 430 forms side wall 436 that extends from intermediary wall 420 to bottom surface 432.

In various embodiments, the first frustoconical aperture 410 is opposite the second frustoconical aperture 430. In other words, the first frustoconical aperture 410 mirrors the second frustoconical aperture 430 about intermediary wall 420. Accordingly, in various embodiments, the combination of the first frustoconical aperture 410 and the second frustoconical aperture 430 form an hour-glass shape.

In one embodiment, the angle 625 of side wall 414 defines an angulation range for a medical instrument (e.g., medical instrument 600) that is penetrated through intermediary wall 420 of plug 400 (see FIG. 5C). In one embodiment, the angle 625 is in a range of 130°-170°.

In one embodiment, intermediary wall 420 is substantially co-planar with top surface 310 of dome 300.

Throughout this specification, reference has been made to a medical device for use in the creation of a temporary pneumoperitoneum in abdominal procedures however it will be understood that the medical device described herein may also be used in other areas where key-hole surgery is performed. The dome-shaped housing may be resized where key-hole surgery is performed. The dome-shaped housing may be resized accordingly so that it will form a suitable seal with the patient's body around the area to be operated upon.

In this specification the terms “comprise, comprises, comprised and comprising” and the terms “include, includes, included and including” are all deemed totally interchangeable and should be afforded the widest possible interpretation.

The invention is in no way limited to the embodiments hereinbefore described but may be varied in both construction and detail within the scope of the claims.

1. A device, comprising:
a hard-body dome comprising:
a first material;
a first outer surface;
an inner surface;
a first grasping wing protruding from the first outer surface, the first grasping wing comprising:
a first upper surface comprising:
a first proximal end proximate the first outer surface; and
a first distal end opposite the first proximal end, wherein the first upper surface protrudes horizontally from the first outer surface from the first proximal end to the first distal end; and
a second outer surface extending substantially vertically from the first distal end to the first outer surface of the hard-body dome;
a second grasping wing protruding from the first outer surface, the second grasping wing comprising:
a second upper surface comprising:
a second proximal end proximate the first outer surface; and
a second distal end opposite the second proximal end, wherein the second upper surface protrudes horizontally from the first outer surface from the first proximal end to the second distal end; and
a third outer surface extending substantially vertically from the second distal end to the first outer surface of the hard-body dome, wherein:
the first grasping wing is circumferentially opposite the second grasping wing; and
the hard-body dome is configured to be placed, by a single hand of a user, at a surface of a patient by:
a first digit of the single hand applying a first downward force on the first upper surface of the first grasping wing; and
a second digit of the single hand concurrently applying a second downward force on the second upper surface of the second grasping wing; and
a first aperture extending from the inner surface to the first outer surface; and
a plug disposed within the first aperture and comprising:
a second material different than the first material;
a third upper surface;
a third lower surface;
an intermediary wall;
a first frustoconical aperture protruding from the third upper surface to the intermediary wall, wherein the first frustoconical opens in the direction from the intermediary wall to the third upper surface; and
a second frustoconical aperture protruding from the third lower surface to the intermediary wall and opposite the first frustoconical aperture, wherein:
the second frustoconical aperture opens in the direction from the intermediary wall to the third lower surface;
the second frustoconical aperture is opposite the first frustoconical about the intermediary wall;
a combination of the first frustoconical aperture and the second frustoconical aperture form a substantially hour-glass shape;
the plug is configured to:
provide a penetrable barrier from the outside surface of the hard-body dome to the inside surface of the hard-body dome; and
allow a medical apparatus to penetrate through the plug and access the surface of the patient disposed within the hard-body dome.

2. The device of claim 1, wherein the hard-body dome is further configured to be removed, by a single hand of the user, from the surface of the patient by:
the first digit of the single hand applying a first upward force on the second outer surface the first grasping wing; and
the second digit of the single hand concurrently applying a second upward force on the second upper surface of the second grasping wing.

3. The device of claim 1, further comprising:
a vacuum feature protruding from the first outer surface of the hard-body dome, the vacuum feature comprising a vacuum aperture extending from a top of the vacuum feature to the inner surface of the hard-body dome, wherein the vacuum aperture is configured to provide a fluid passageway from within the hard-body dome to an outside of the hard-body dome.

4. The device of claim 3, wherein the vacuum feature comprises a conical shape.

5. The device of claim 3, wherein the vacuum feature protrudes vertically from the first outer surface.

6. The device of claim 3, wherein a fourth upper surface of the vacuum feature is substantially co-planar with the third upper surface of the plug.

7. The device of claim 1, wherein:
the second outer surface of the first grasping wing comprises a first grip feature protruding from the second outer surface; and
the third outer surface of the second grasping wing comprises a second grip feature protruding from the third outer surface.

8. The device of claim 1, wherein:
the second outer surface of the first grasping wing comprises a first grip aperture that extends into the second outer surface; and
the third outer surface of the second grasping wing comprises a second grip aperture that extends into the third outer surface.

9. A device, comprising:
a hard-body dome comprising:
a first material;
a first outer surface;
an inner surface;
a first grasping wing protruding horizontally from the first outer surface of the hard-body dome;
a second grasping wing protruding horizontally from the first outer surface of the hard-body dome, wherein the second grasping wing is opposite the first grasping wing, wherein:
the hard-body dome is configured to be placed, by a single hand of a user, at a surface of a patient by:
a thumb of the single hand applying a first downward force on a first upper surface of the first grasping wing; and
a finger of the single hand concurrently applying a second downward force on a second upper surface of the second grasping wing; and
a vacuum aperture extending from the first outer surface to the inner surface, wherein a suction force applied through the vacuum aperture draws the surface of the patient within the hard-body dome, while the hard-body dome is placed by the single hand of the user.

10. The device of claim 9, wherein the hard-body dome is further configured to be separated, by the single hand of the user, from the surface of the patient by:
the thumb of the single hand applying a first upward force on a first bottom surface of the first grasping wing; and
the finger of the single hand concurrently applying a second upward force on a second bottom surface of the second grasping wing.

11. The device of claim 9, wherein:
the first grasping wing comprises:
a first lateral wall extending vertically from the first upper surface to the first outer surface of the hard-body dome; and
a second lateral wall opposite the first lateral wall extending vertically from the first upper surface to the first outer surface of the hard-body dome; and
the second grasping wing comprises:
a third lateral wall extending vertically from the second upper surface to the first outer surface of the hard-body dome; and
a fourth lateral wall opposite the third lateral wall extending vertically from the second upper surface to the first outer surface of the hard-body dome.

12. The device of claim 9, wherein the first grasping wing and the second grasping wing are comprised of the first material or a second material different than the first material.

13. The device of claim 9, further comprising:
a first distance between an apex of the hard-body dome and the first grasping wing; and
a second distance between the apex of the hard-body dome and the second grasping wing, wherein the first distance is the same as the second distance.

14. The device of claim 9, further comprising:
a line bisecting the first grasping wing, the second grasping wing and an apex of the hard-body dome, wherein the vacuum aperture is laterally offset from the line.

15. A device, comprising:
a hard-body dome comprising:
a first material;
an outer surface;
an inner surface;
a flat upper surface comprising a first aperture extending from the inner surface to the outer surface;
a plug disposed within the first aperture and comprising:
a second material different than the first material;
an upper surface;
a bottom surface opposite the upper surface;
an intermediary wall;
a first frustoconical aperture protruding from the upper surface to the intermediary wall and defining a first lateral wall in the plug extending from the upper surface to the intermediary wall; and
a second frustoconical aperture protruding from the bottom surface to the intermediary wall and opposite the first frustoconical aperture and defining a second lateral wall in the plug extending from the bottom surface to the intermediary wall, wherein:
the first frustoconical aperture faces an opposite direction than the second frustoconical aperture;
a combination of the first frustoconical aperture and the second frustoconical aperture form an hour-glass shape; and
the plug is configured to:
provide a penetrable barrier from the outer surface of the hard-body dome to the inner surface of the hard-body dome; and
allow a medical apparatus to:
penetrate through the plug at an access point;
rotate circumferentially about the access point; and
pivot about the access point within an angulation range defined by the first lateral wall and the second lateral wall.

16. The device of claim 15, wherein the angulation range prohibits the medical apparatus to contact the inner surface of the hard-body dome.

17. The device of claim 15, wherein the first material is an impermeable material and the second material is a semi-permeable material.

18. The device of claim 15, wherein the angulation range is up to 150°.

19. The device of claim 15, wherein the intermediary wall of the plug is co-planar with the flat upper surface of the hard-body dome.

20. The device of claim 15, wherein the upper surface of the plug is vertically offset from the flat upper surface of the hard-body dome.
